(19) United States
(12) Patent Application Publication
(10) Pub. No.: US 2010/0074896 A1
(43) Pub. Date: Mar. 25, 2010

(54) ANTAGONISTS OF PGE2 EP3 RECEPTORS

(75) Inventors: Sylvain Dore, Baltimore, MD (US); Abdullah Ahmad, Owings Mills, MD (US)

Correspondence Address: BANNER & WITCOFF, LTD., 1100 13th STREET, N.W., SUITE 1200, WASHINGTON, DC, 20005-4051, US

(73) Assignee: The Johns Hopkins University, Baltimore, MD (US)

(21) Appl. No.: 12/516,847

(22) PCT Filed: Nov. 30, 2007
(86) PCT No.: PCT/US07/86068

§ 371 Date: Nov. 9, 2009

Related U.S. Application Data
(60) Provisional application No. 60/861,816, filed on Nov. 30, 2006.

(51) Int. Cl. A61K 39/395

(52) U.S. Cl. 424/133.1; 424/130.1

(57) ABSTRACT

PGE2 EP3 receptors affect injury size following cerebral ischemia and induced excitotoxicity. Treatment with selective EP3 antagonists decreases infarct size. In addition, such antagonists can reduce lesions caused by N-methyl-D-aspartic acid-induced acute excitotoxicity. Similarly, genetic deletion of EP3 provides protection against N-methyl-D-aspartic acid-induced toxicity. PGE2, by stimulating EP3 receptors, can contribute to the toxicity associated with cyclooxygenase and that antagonizing this receptor can be used therapeutically to protect against stroke- and excitotoxicity-induced brain damage.

The present invention was made using funds from the United States government. The United States retains certain rights in the invention according to the terms of National Institutes of Health grants NS046400 and AG022971.

### TECHNICAL FIELD OF THE INVENTION

This invention is related to the area of chronic and acute neurodegenerative diseases. In particular, it relates to treatment and prevention of such diseases.

### BACKGROUND OF THE INVENTION

Inflammation has been shown to play a major role in the pathological response and outcome of stroke and other central nervous system disorders (Huang et al., 2006; Lucas et al., 2006). Inflammation is mediated at least in part by prostaglandins (PGs), which are produced through the cyclooxygenase (COX) pathway. PGs are secreted from a variety of cells in response to physiological or pathological insults (Doré et al., 2003; Minghetti, 2004) and mediate a variety of actions via specific membrane-bound receptors to maintain local homeostasis. Prostaglandin E2 (PGE₂) mainly binds to a family of G-protein-coupled receptors known as EP receptors (Narumiya et al., 1999). The members of the EP receptor family, EP1, EP2, EP3, and EP4, elicit their actions by altering cyclic adenosine monophosphate (cAMP) or intracellular calcium concentrations. EP1 activates phospholipase C and phosphatidylinositol turnover and stimulates the release of intracellular calcium via a Gi-coupled mechanism. EP2 and EP4 both signal through a Gs-coupled mechanism that stimulates adenylyl cyclase and increases intracellular levels of cAMP (Narumiya et al., 1999). The EP3 receptor, which has several isoforms (Bilson et al., 2004), mediates the activation of several signaling pathways, leading to changes in cAMP levels, calcium mobilization, and activation of phospholipase C (Namba et al., 1993; Narumiya et al., 1999). Of the three isoforms identified in mouse, EP3α and EP3β are reported to be coupled to Gi protein, which leads to the inhibition of adenylyl cyclase, whereas EP3γ has both inhibitory and stimulatory effects on adenylyl cyclase and cAMP accumulation (Irie et al., 1994; Sugimoto et al., 1993). Eight isoforms of EP3 have been reported in humans (Amano 2003, Hayashi 2007, and Aihara 2007).

It has been reported that EP3 receptors are expressed in glial cells after intrastriatal injection of quinolic acid in rats (Slawik et al., 2004). This finding implies a direct role for EP3 receptors in various neurodegenerative disorders, such as stroke and Alzheimer disease. In addition, Zacharowski and colleagues (Zacharowski et al., 1999) reported that ONO-AE-248, a selective EP3 agonist, prevented the forskolin-induced increase in cAMP in CHO cell lines. Recently, Yamazaki et al. (2005) showed that EP3 receptor protein expression was significantly elevated in placenta 24 h after ischemia-reperfusion injury. EP3 receptors also have been shown to participate in inflammatory reactions in a mouse model of pleurisy, a model of acute inflammation (Yuhki et al., 2004), and have been reported to trigger pulmonary edema induced by platelet-activating factor in rats (Goggel et al., 2002). In addition, activation of EP3 receptor by PGE₂ in mice inhibits cAMP production in platelets and promotes platelet aggregation (Fabre et al., 2001).

We previously reported that in mice, the EP1 receptor plays a toxic role in transient cerebral ischemia and excitotoxicity models (Ahmad et al., 2006a), a finding further substantiated by Kawano et al. (2006), and that EP2 and EP4 receptor activation is protective in N-methyl-D-aspartic acid (NMDA)-induced excitotoxic lesions (Ahmad et al., 2005; Ahmad et al., 2006b). We also have evaluated the effects of the drug 1-OH-PGE₁, which stimulates EP4 and to a lesser extent EP3, and found it to be neuroprotective in transient ischemia (Ahmad et al., 2006c) and oxidative stress after β-amyloid exposure in mouse primary cultured neurons (Echeverria et al., 2005).

There is a continuing need in the art to identify agents which can reduce the risk of acute ischemic/excitotoxic events and for agents which can treat or ameliorate the effects of acute or chronic neurodegenerative diseases.

### SUMMARY OF THE INVENTION

According to one embodiment of the invention a method is provided for treating a patient with an acute or chronic neurodegenerative disease. An antagonist of an EP3 receptor is administered to the patient, whereby a symptom of the disease is ameliorated.

According to another embodiment of the invention a method is provided for reducing the risk of an ischemic hypoxic reperfusion event in a subject. An antagonist of an EP3 receptor is administered to a subject at increased risk of having a stroke, whereby the risk of hypoxic/excitotoxic event is reduced.

These and other embodiments which will be apparent to those of skill in the art upon reading the specification provide the art with methods for treating, ameliorating and preventing ischemic hypoxic events and other acute or chronic neurodegenerative diseases.

### BRIEF DESCRIPTION OF THE DRAWINGS

FIGS. 1A and 1B: Schematic representation of the protocol for middle cerebral artery occlusion surgery (FIG. 1A) and analysis of physiological parameters (FIG. 1B).

FIG. 2A-2C. Effect of the EP3 receptor agonist ONO-AE-248 on percent corrected hemispheric infarct volumes in a mouse model of middle cerebral artery (MCA) occlusion. FIG. 2A: Western blot showing the presence of an immunoreactive profile corresponding to the estimated molecular weight of the mouse EP3 receptors in corticostriatal mouse brain homogenate. Mice were given ONO-AE-248 or vehicle in the lateral ventricle before being subjected to 90 min of occlusion and 4 days of reperfusion.

FIG. 2B: Representative photographs of TTC-stained sections of mouse brain that were injected either with vehicle (left panel) or 5 nmol ONO-AE-248 (right panel) followed by MCAO and reperfusion. The unstained areas indicate the area of infarction. FIG. 2C: Hemispheric infarct volumes were significantly larger in mice treated with 2.5 and 5.0 nmol ONO-AE-248 than in vehicle-treated mice. The results are expressed as mean±S.D. of 9 animals per group. *p<0.05 compared with the vehicle-treated group.

FIG. 3. Core body temperature was recorded with an intra-abdominal radiofrequency probe every 10 min during the first 90 min after ONO-AE-248 injection and then once daily for 4 days while the mice were housed at room temperature. No significant differences in core body temperature were observed at any dose of ONO-AE-248 as compared with those of the vehicle-treated group. The results are expressed as mean±S.D. of 5 animals per group.

FIG. 4A-4B. Effect of EP3 receptor selective agonist ONO-AE-248 on NMDA-induced brain lesion in C57B1/6 WT mice. FIG. 4A: Representative photographs of sections of mouse brain that were injected with either vehicle (left panel) or 5 nmol ONO-AE-248 (right panel) followed by NMDA, and stained with Cresyl Violet. FIG. 4B: Histograms representing volume of NMDA-induced brain injury. ONO-AE-248 significantly increased the injury volume observed in the groups pretreated with 2.5 nmol and 5.0 nmol ONO-AE-248 (n=7/dose); however, no difference in brain lesion was observed at the lowest dose of 0.5 nmol as compared with NMDA alone (15 nmol, n=7). Values are reported as means±S.D.*p<0.02, **p<0.001 when compared with the group given NMDA alone. Scale bar=1000 μm.

FIG. 5A-5B. NMDA-induced toxicity in the brains of C57B1/6 WT and EP3^{-/-} mice. A single dose of NMDA (15 nmol) was injected into the right striatum of C57B1/6 WT (n=7) and EP3^{-/-} mice (n=8). FIG. 5A: Representative photographs of mouse brain sections injected with NMDA and stained with Cresyl Violet. FIG. 5B: Quantitative analysis showed that the lesion volume in EP3^{-/-} mice was significantly smaller than that in WT mice. The lesion volume was attenuated by 26±10% as compared with the WT mice. Values are reported as means±S.D.*p<0.01. Scale bar=1000 μm.

### DETAILED DESCRIPTION OF THE INVENTION

The inventors have developed a method for treating subjects at elevated risk of an ischemic hypoxic event or who have other acute or chronic neurodegenerative diseases. The method involves the administration to a subject of an antagonist of an EP3 receptor.

Abbreviations used in this application include: cAMP, cyclic adenosine monophosphate; CBF, cerebral blood flow, PG, prostaglandin; MABP, mean arterial blood pressure; MCA, middle cerebral artery; NMDA, N-methyl-d-aspartic acid; TTC, 2,3,5-triphenyl-tetrazolium chloride; ICV, intracerebroventricular.

Subjects who may be treated include mammals of all types which are subject to acute or chronic neurodegenerative diseases. These include without limitation, rats, mice, pigs, cows, dogs, cats, rabbits, monkeys, chimpanzees. The subjects can be pets, farm animals, laboratory models for human disease, or humans. The subjects may be afflicted with a neurodegenerative disease, prone to an acute or chronic neurodegenerative disease, or prone to ischemia. The tendency to ischemia may result from pharmacological or surgical intervention. The tendency to ischemia or neurodegenerative disease may result from genetic predisposition.

Diseases which may be treated or prevented (which includes a lessening of the risk of occurrence) include without limitation diseases such as Alzheimer's disease, Parkinson's disease, Huntington's disease, ischemic stroke, hemorrhagic stroke, global ischemia, head trauma, age-related vascular dementia, cognitive disorders.

Treatment includes any detectable improvement in symptoms or disease associated pathology. These may be detected by any means known in the art, including patient self-assessment, physician assessment, radiological assessment, histopathological assessment, biochemical assessment, neurological assessment, cognitive assessment, sensory assessment, speech assessment, affect assessment.

Prevention includes the statistically ascertained reduction in risk. This need not be a total or absolute prevention, but rather a lessening in the risk of the event. Reduction in risk is typically ascertained for groups of subjects who are stratified according to certain defined characteristics. Changing such characteristics in an individual is presumed to reduce the risk for that individual, because that individual would then fall within a group with a reduced risk of the event.

Antagonists of an EP3 receptor include any compound, whether small molecule or biological, which prevents prostaglandin E₂ from having its normal biological effect, i.e., receptor signalling. The EP3 receptors couple to Gi-type G protein, which leads to a decrease of intracellular cAMP levels and increased intracellular calcium levels. The antagonist can act on any of the eight splice variant forms of EP3 receptor or isoforms. Antagonists include but are not limited to L798106, ONO-AE3-208 (2-[[2-[2-(2-methylnaphthalen-1-yl)propanoylamino]phenyl]methyl]benzoic acid), ONO-AE3-240, ONO8711, SC-51322. See also, Singh et al., US 2006/0142355, US 2006/0142348, the disclosures of which are expressly incorporated herein.

The receptor isoforms are variously called EP3A, EP3-I, EP3a1, EP3a2 , EP3C, EP3-II EP3B, EP3-III, EP3D, EP3-IV, EP3F, EP3E, EP3F, and EP3G. The receptors are expressed at least in small intestine, heart and pancreas. Antagonists of all or one or more of the isoforms may be used.

Antagonists of EP3 receptors can be determined by any means known in the art. Competition of PGE₂ binding, for example can be determined using a [³H]PGE₂ binding assay. Cell membranes can be prepared and incubated with tritiated PGE₂. Nonspecific binding can be determined using excess unlabeled PGE₂. Specific binding can be calculated by subtracting the nonspecific binding from total binding. Alternatively or additionally, intracellular free Ca²⁺ concentration ([Ca²⁺]ᵢ) can be determined. The [Ca²⁺]ᵢ can be measured as described in Miwa, et al (1988) J. Neurochem. 50, 1418-1424. Fluorescence can be measured at excitation wavelengths of 340 and 380 nm and an emission wavelength of 510 nm, with a fluorescence spectrometer. The [Ca²⁺]ᵢ can be calculated from cellular fura-2 fluorescence. Alternatively or additionally cAMP formation can be measured as reported in Okuda-Ashitaka, et al. (1990) Eicosanoids 3, 213-218. The cAMP formed can be measured using a radioimmunoassay such as an Amersham cAMP assay kit. Antagonists will have at least a 5% reducing effect on a measured parameter. Preferred antagonists will have at least a 10, 15, 20, 25, 30, 45, 50, 55, 60, 65, 70, 75, 80, 85, 90, or 95% reducing effect on a measured parameter.

Delivery of an antagonist can be by any means known in the art. Often in the case of ischemic stroke, the blood brain barrier is disrupted and drugs can more easily access the brain. If the blood brain barrier is intact, then intracerebroventricular injection may be used. Some antagonists are able to cross the barrier, even when intact (e.g., EP3-P). Any other form of delivery should also be considered for its ease and efficiency, such as per os, intravenous, intraperitoneal, topical, subcutaneous, intradermal, sublingual, etc.

Antibodies can be used as antagonists, as is known in the art. Such can be polyclonal or monoclonal, chimeric, humanized, or human. Fragments of antibodies can be used, as can recombinant antibody-like constructs, such as single-chain antibodies. Antibodies to peptide NH₂-IFNQTSVEMCKTHTEK—COOH (SEQ ID NO: 1), which is common to all isoforms of EP3 receptor, have been raised in rabbits. Morath et al., J. Am Soc Nephrol. 10: 1851-1860, 1999. Commercially available mouse monoclonal antibody is reactive with human, rat, cow, and pig EP3. AbCam Plc. Other commercial suppliers of anti-EP3 antibodies include ABR-Affinity BioReagents, Aviva Systems Biology, Cayman Chemical, Everest Biotech, Exalpha Biologicals, Inc., GeneTex, GenWay Biotech, Inc., IMGENEX, MBL International, Millipore Corporation, Novus Biologicals, Santa Cruz Biotechnology, Inc., and Sigma-Aldrich. These and other antibodies and antibody fragments can be routinely tested for antagonist effect using standard assays.

Pharmaceutical compositions can comprise, for example, an antagonist of EP3 receptors which specifically binds to EP3 receptors and prevent or reduce the effect of an EP3 agonist. The compositions can be administered alone or in combination with at least one other agent, such as stabilizing compound, which can be administered in any sterile, biocompatible pharmaceutical carrier, including, but not limited to, saline, buffered saline, dextrose, and water. The compositions can be administered to a patient alone, or in combination with other agents, drugs or hormones.

In addition to the active ingredients, these pharmaceutical compositions can contain suitable pharmaceutically-acceptable carriers comprising excipients and auxiliaries that facilitate processing of the active compounds into preparations which can be used pharmaceutically. Pharmaceutical compositions of the invention can be administered by any number of routes including, but not limited to, oral, intravenous, intramuscular, intra-arterial, intramedullary, intrathecal, intraventricular, transdermal, subcutaneous, intraperitoneal, intranasal, parenteral, topical, sublingual, or rectal means. Pharmaceutical compositions for oral administration can be formulated using pharmaceutically acceptable carriers well known in the art in dosages suitable for oral administration. Such carriers enable the pharmaceutical compositions to be formulated as tablets, pills, dragees, capsules, liquids, gels, syrups, slurries, suspensions, and the like, for ingestion by the patient.

Pharmaceutical preparations for oral use can be obtained through combination of active compounds with solid excipient, optionally grinding a resulting mixture, and processing the mixture of granules, after adding suitable auxiliaries, if desired, to obtain tablets or dragee cores. Suitable excipients are carbohydrate or protein fillers, such as sugars, including lactose, sucrose, mannitol, or sorbitol; starch from corn, wheat, rice, potato, or other plants; cellulose, such as methyl cellulose, hydroxypropylmethyl-cellulose, or sodium carboxymethylcellulose; gums including arabic and tragacanth; and proteins such as gelatin and collagen. If desired, disintegrating or solubilizing agents can be added, such as the cross-linked polyvinyl pyrrolidone, agar, alginic acid, or a salt thereof, such as sodium alginate.

Dragee cores can be used in conjunction with suitable coatings, such as concentrated sugar solutions, which also can contain gum arabic, talc, polyvinylpyrrolidone, carbopol gel, polyethylene glycol, and/or titanium dioxide, lacquer solutions, and suitable organic solvents or solvent mixtures. Dyestuffs or pigments can be added to the tablets or dragee coatings for product identification or to characterize the quantity of active compound, i.e., dosage.

Pharmaceutical preparations that can be used orally include push-fit capsules made of gelatin, as well as soft, sealed capsules made of gelatin and a coating, such as glycerol or sorbitol. Push-fit capsules can contain active ingredients mixed with a filler or binders, such as lactose or starches, lubricants, such as talc or magnesium stearate, and, optionally, stabilizers. In soft capsules, the active compounds can be dissolved or suspended in suitable liquids, such as fatty oils, liquid, or liquid polyethylene glycol with or without stabilizers.

Pharmaceutical formulations suitable for parenteral administration can be formulated in aqueous solutions, preferably in physiologically compatible buffers such as Hanks' solution, Ringer's solution, or physiologically buffered saline. Aqueous injection suspensions can contain substances that increase the viscosity of the suspension, such as sodium carboxymethyl cellulose, sorbitol, or dextran. Additionally, suspensions of the active compounds can be prepared as appropriate oily injection suspensions. Suitable lipophilic solvents or vehicles include fatty oils such as sesame oil, or synthetic fatty acid esters, such as ethyl oleate or triglycerides, or liposomes. Non-lipid polycationic amino polymers also can be used for delivery. Optionally, the suspension also can contain suitable stabilizers or agents that increase the solubility of the compounds to allow for the preparation of highly concentrated solutions. For topical or nasal administration, penetrants appropriate to the particular barrier to be permeated are used in the formulation. Such penetrants are generally known in the art.

The pharmaceutical compositions of the present invention can be manufactured in a manner that is known in the art, e.g., by means of conventional mixing, dissolving, granulating, dragee-making, levigating, emulsifying, encapsulating, entrapping, or lyophilizing processes. The pharmaceutical composition can be provided as a salt and can be formed with many acids, including but not limited to, hydrochloric, sulfuric, acetic, lactic, tartaric, malic, succinic, etc. Salts tend to be more soluble in aqueous or other protonic solvents than are the corresponding free base forms. In other cases, the preferred preparation can be a lyophilized powder which can contain any or all of the following: 1-50 mM histidine, 0.1%-2% sucrose, and 2-7% mannitol, at a pH range of 4.5 to 5.5, that is combined with buffer prior to use.

Further details on techniques for formulation and administration can be found in the latest edition of REMINGTON'S PHARMACEUTICAL SCIENCES (Maack Publishing Co., Easton, Pa.). After pharmaceutical compositions have been prepared, they can be placed in an appropriate container and labeled for treatment of an indicated condition. Such labeling would include amount, frequency, and method of administration.

There have been some conflicting reports in the literature in regard to the role of EP3 receptors in health and disease; some investigators have reported that EP3 affords protection whereas others state that it aggravates toxicity. Considering that PGE₂ is often called the “pro-inflammatory” prostaglandin, we have used the mouse models of cerebral ischemia and induced excitotoxicity to elucidate a better understanding of the function of the EP receptors and help resolve some of the contradictory findings. Our current study was designed to determine the role of EP3 in acute excitotoxicity and stroke. We found that 2.5- and 5.0-nmol doses of the selective EP3 receptor agonist ONO-AE-248 administered ICV significantly exacerbated the lesion volume following NMDA injection and the infarct volume following transient ischemia, while none of the physiological parameters studied were significantly affected. We also found that genetic deletion of the EP3 receptor reduced the resulting lesion size after NMDA injection as compared to that observed in WT littermates. To our knowledge this is the first report to show that stimulation of the EP3 receptors in the brain exacerbates brain damage.

COX-2 is constitutively present at low levels but is highly inducible and suggested to be responsible for inflammatory reactions (Morham et al., 1995). Several groups have documented the induction of COX-2 after cerebral ischemia (Collaco-Moraes et al., 1996; Planas et al., 1995; Tomimoto et al., 2002). It is well established that pharmacologic inhibition of COX-2 activity and genetic deletion of COX-2 (Iadecola et al., 2001) are protective in cerebral ischemia, whereas COX-2 overexpression in a transgenic model is detrimental (Doré et al., 2003). However, when COX-2 was overexpressed in neurons of mice, we found that COX-2 inhibitors could not decrease the size of infarcts caused by cerebral ischemia (Doré et al., 2003). Moreover, recent clinical reports of the side effects associated with the COX-2 inhibitors led us to consider that a better strategy for treating ischemic injury might lay in modulating the PGs and PG receptors downstream of the COX pathway.

PGE₂ has been reported to execute its toxic actions mainly via the EP3 receptor in a rat model of passive Heymann nephritis (Waldner et al., 2003). Furthermore, EP3 receptor and COX-2 immunoreactivity have been reported to colocalize and increase in rat placenta after ischemia-reperfusion of the uterine artery (Yamazaki et al., 2005). Therefore, it seems very likely that the EP3 receptor would be involved in ischemia-reperfusion injury in the brain.

We found that ICV injection of ONO-AE-248 did not produce detectable changes in the various physiological parameters monitored. In earlier studies, stimulation of the EP3 receptor with 17-phenyl trinor PGE₂, sulprostone (EP1/EP3 agonists) (Jadhav et al., 2004) or ONO-AE-248 (Norel et al., 2004) was reported to cause vasoconstriction, but we did not observe significant changes in MABP at any dose of ONO-AE-248 tested under our experimental conditions. One possibility for this finding is that the effects might have been too short-lived or not large enough to be measured. Alternatively, the dissimilar results could stem from the use of different models; the latter studies were carried out in vitro in arteries dissected from either pig cerebral arteries (Jadhav et al., 2004) or human pulmonary arteries (Norel et al., 2004), whereas our experiments were conducted in vivo. Furthermore, 17-phenyl trinor PGE₂ and sulprostone have high affinities for the EP1 receptor (14 and 4 nM, respectively) in addition to the EP3 receptor (21 and 0.6 nM, respectively), whereas ONO-AE-248 is highly selective toward EP3 receptor. In fact, ONO-AE-248 is estimated to be 1333 times more specific for EP3 than for EP1 (Kiriyama et al., 1997; Narumiya and FitzGerald, 2001; Suzawa et al., 2000).

Assessment of CBF by laser-Doppler flowmetry indicated that MCAO resulted in similar decreases in cortical perfusion throughout the ischemic period in vehicle-treated and drug-treated mice, suggesting that the mechanism by which EP3 affects the severity of the ischemic insult was not by altering blood flow with its stimulation by ONO-AE-248. These results predict a toxic role for EP3 by mechanisms other than increasing the ischemic insult.

The core body temperature was not significantly affected by ONO-AE-248 at any time during our experimental protocol. Although this result might appear to be in conflict with that of a previous study that showed that mild hyperthermia was induced after a single ICV injection of ONO-AE-248 in rats (Oka, 2004), in our experimental design, the temperature of the animals was strictly maintained at 37.0±0.5° C. To further address the potential effect of ONO-AE-248 alone on temperature, we treated a separate cohort of animals that were allowed free movement at room temperature. Like the mice that underwent the MCAO, these mice showed no changes in temperature. Accordingly, we believe that our results are accurate, but may differ from the previous study, which used rats and a higher dose (20 nmol ICV) of ONO-AE-248.

Stimulation of EP3 receptors is known to affect the release of intracellular calcium reserves. The increase in intracellular calcium can activate many enzymes, such as phospholipase C, phospholipase A2, and neuronal nitric oxide synthase. Each of these enzymes can increase stroke injury, and their inhibition has been shown to confer protection (Lipton, 1999). Abnormal calcium accumulation is also reported to cause mitochondrial dysfunction (Atlante et al., 2001) by depolarizing mitochondrial membrane potential (Akerman, 1978; Loew et al., 1994) and reducing ATP synthesis, which is thought to be a primary cause of cell death (Schinder et al., 1996). Reduction in ATP synthesis caused by increases in calcium concentration can lead to enhanced generation of reactive oxygen species and eventually to neurotoxicity (Antonsson et al., 1997; Bauer et al., 1998; Green and Reed, 1998; Zamzami et al., 1995).

Cyclic AMP elicits a wide range of cellular functions; it is reported to be involved in neuronal survival, axonal regeneration, and enhancement of neurite outgrowth (Hansen et al., 2001; Kao et al., 2002; Rydel and Greene, 1988). Activation of the EP3 receptor has been shown to inhibit cAMP synthesis in murine platelets (Fabre et al., 2001). In our study, activation of EP3 receptor by ONO-AE-248 might have inhibited cAMP synthesis and subsequently might have blocked the related downstream signaling pathways, such as protein kinase A and extracellular signal-regulated kinase, which are involved in cell survival. It is likely that the inhibition of these pathways enhanced cerebral injury, though further work in warranted to better address the exact signaling pathway involved in the toxicity mediated through EP3 receptors.

It is of interest that pharmaceutical companies are also considering the use of EP3 antagonists for clinical use. For example, a selective and potent EP3 receptor antagonist, DG041 (deCode genetics, 2006), is undergoing Phase-IIa clinical trials for peripheral arterial disease, which is characterized early by symptoms of pain or fatigue in the legs and buttocks during activity. People suffering with this disease have a higher than normal risk of death from heart attack and stroke. Clinical trials showed that oral administration of DG041 was well-tolerated, with no serious drug-related adverse events noted. Such results provide promise that EP3 mimetic drugs could be developed for use in other disorders, such as ischemia-reperfusion injury and tested in pre-clinical models and clinical settings.

In conclusion, since activation of EP3 receptors in brain worsened stroke outcome and excitotoxic brain lesion, and deletion of the receptor improved the outcome after NMDA-induced excitotoxicity and transient cerebral ischemia, we propose that deletion or pharmacologic blockade of this receptor might limit brain damage induced by pro-inflammatory COX metabolites and that these receptors could be used as therapeutic targets for the prevention or reduction of ischemia-reperfusion-associated brain injury.

The above disclosure generally describes the present invention. All references disclosed herein are expressly incorporated by reference. A more complete understanding can be obtained by reference to the following specific examples which are provided herein for purposes of illustration only, and are not intended to limit the scope of the invention.

### EXAMPLE 1

### Materials and Methods

### Animals and Drugs

Studies were carried out on 8- to 10-week-old male C57BL/6 mice weighing 25 to 30 g obtained from Charles River Laboratories, Inc (Wilmington, Mass.). EP3 receptor knockout (EP3^{-/-}) mice were provided by Shuh Narumiya, University of Kyoto, Japan, and genotypes were confirmed by PCR. All animal protocols were approved by the Johns Hopkins University Animal Care and Use Committee. The animals were allowed free access to water and food before and after surgery. ONO-AE-248 was kindly donated by ONO Pharmaceuticals (Osaka, Japan).

### Assessment of EP3 Receptor Protein Expression in Mouse Brain

To address whether EP3 is present in mouse cortex and striatum, homogenates of the corticostriatal region of mouse brains were analyzed by Western blot, as described previously (Ahmad et al., 2006c). Protein concentrations were quantified by BCA assay (Pierce, Rockford, Ill.). Electrophoresis was performed on 12% polyacrylamide gels (Invitrogen, Carlsbad, Calif.), and proteins were transferred to nitrocellulose membrane (BIO-RAD, Hercules, Calif.). Blots were stained with Ponceau S Solution (Sigma, St. Louis, Mo.) to verify that equal amounts of protein were loaded into each lane. Membranes were blocked for 1 h at room temperature with 5% skim milk in phosphate-buffered saline with 0.1% Tween 20 before being incubated at 4° C. overnight with rabbit EP3 polyclonal antibody (1:500; Cayman, Ann Arbor, Mich.). Blots were washed and incubated with secondary antibody for 1 h at room temperature and then developed with ECL (Amersham Biosciences, Piscataway, N.J.).

### Stereotactic Injection

Mice were anesthetized with 3.0% halothane and maintained with 1.0-1.5% continuous flow of halothane in oxygen-enriched air. Then the mice were mounted on a stereotactic frame and injected with 0.2 μl of different doses of ONO-AE-248 (0.5 nmol, 2.5 nmol, or 5.0 nmol) or vehicle (DMSO) via a 1μl Hamilton syringe (Reno, Nev.) into the right lateral ventricle as described previously (Ahmad et al., 2006a). After the injection, the needle was retracted slowly, the hole was plugged with bone wax, and the wound was sutured. Mice were then either transferred to another setup for the MCAO procedure or left in place for NMDA injection (see below).

### MCAO and Reperfusion

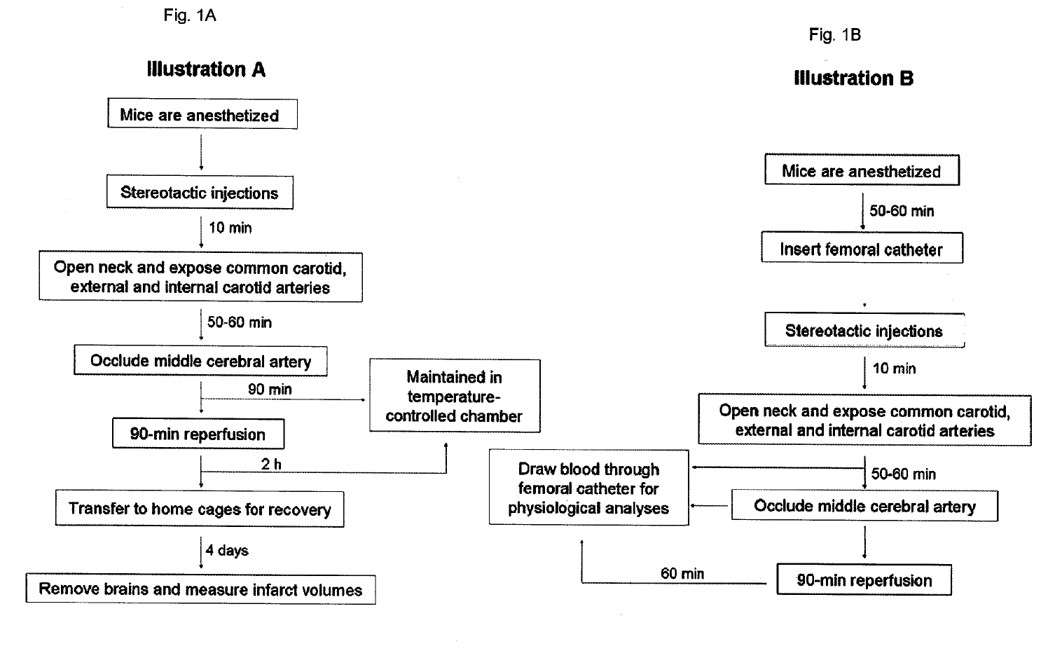

During the MCAO procedure (FIG. 1, Illustration A), mouse rectal temperature was monitored and maintained at 37.0±0.5° C. by a heating pad, and anesthesia was maintained with continuous flow of halothane in oxygen-enriched air via a nose cone. Relative CBF was monitored with laser-Doppler flowmetry (Moor Instruments, Devon, England) by a flexible fiber optic probe affixed to the skull over the parietal cortex supplied by the MCA (2 mm posterior and 6 mm lateral to the bregma). MCAO was carried out under aseptic conditions with a silicone-coated nylon monofilament as described previously (Shah et al., 2006). The filament was left in position for 90 min, during which the incision was closed with sutures, anesthesia was discontinued, and the animals were transferred to a temperature-controlled chamber that maintains the body temperature of mice at 37.0±0.5° C. At 90 min of occlusion, the mice were briefly reanesthetized with halothane, and reperfusion was achieved by withdrawing the filament and reopening the MCA. After the incisions were sutured, the mice were returned to the temperature-controlled chamber for 2 h before being transferred to their home cages for 4 days.

### Quantification of Infarct Volume

Four days after MCAO surgery, mice (n=9/dose) were deeply anesthetized, and their brains were harvested and sliced coronally into five 2-mm-thick sections. The sections were incubated with 1% 2,3,5-triphenyl-tetrazolium chloride (TTC) in saline for 20 min at 37° C. The area of infarcted brain, identified by the lack of TTC staining, was measured on the rostral and caudal surfaces of each slice and numerically integrated across the thickness of the slice to obtain an estimate of infarct volume in each slice (SigmaScan Pro, SPSS, Port Richmond, Calif., USA). Volumes from all five slices were summed to calculate total infarct volume over the entire hemisphere and expressed as a percentage of the volume of the contralateral structure. Infarct volume was corrected for swelling by comparing the volumes in the ipsilateral and contralateral hemispheres. The corrected infarct volume was calculated as: volume of contralateral hemisphere−(volume of ipsilateral hemisphere−volume of infarct).

### Analysis of Physiological Parameters

Measurement of physiological parameters was carried out in a group of mice separate from that used to assess infarct volume (FIG. 1, Illustration B). Arterial blood samples collected via femoral catheter were analyzed for pH, PaO₂, and PaCO₂ before the occlusion, during occlusion, and 1 h after the occlusion. MABP was measured at the same time points by a pressure transducer connected to the femoral catheter.

### Temperature Regulation

A third cohort of mice was implanted with intra-abdominal radiofrequency probes [IPTT-200, Bio. Medic. Data System (BMDS), Seaford, Del.] 7 days before injection of ONO-AE-248 (n=5) or vehicle (n=5). Core temperature was sampled every 10 min for the first 90 min after injection, and then once daily for 4 days at room temperature via receivers (The DAS-5002 Notebook System™; BMDS). This telemetry system minimizes stress and allows temperature control and monitoring in freely moving animals.

### NMDA Injection

To investigate the effect of NMDA toxicity in mice lacking the EP3 receptor, WT (n=7) and EP3-/- (n=8) mice were injected in the right striatum with 15 nmol NMDA or vehicle in a volume of 0.3 μl 20 min after being injected with ONO-AE-248, as reported earlier (Ahmad et al., 2006a). After injection, the hole was blocked and the wound sutured, as described above. After the surgical procedures, the animals were transferred to a temperature-regulated chamber to recover from anesthesia. Throughout the stereotactic procedures the rectal temperature of mice was monitored and maintained at 37.0±0.5° C.

### Quantification of NMDA-Induced Lesion Volume

At 48 h after NMDA injection, weight and rectal temperature of each mouse was recorded. Then, mice were transcardially perfused with phosphate-buffered saline, followed by 4% paraformaldehyde (pH 7.2), under deep anesthesia. Brains were immediately removed, post-fixed in paraformaldehyde overnight, cryoprotected in sucrose (30%) for 3 days, and frozen in 2-methyl butane (pre-cooled over dry ice). Brain sections cut on a cryostat were collected on microscope slides and stained with Cresyl Violet to estimate lesion volume. Images of the brain sections were taken and analyzed with SigmaScan Pro 5.0 software (Systat Software Inc., Richmond, Calif.) as described previously (Ahmad et al., 2006b).

### Statistical Analysis

Data were analyzed with SigmaStat 2.0 (Systat Software Inc.). One-way ANOVA followed by Tukey's post-hoc analysis was used to calculate the difference between the groups. Core body temperature was analyzed by two-way ANOVA followed by the Neuman-Keuls test. Significance level was set at p<0.05.

### EXAMPLE 2

### Presence of EP3 Receptor Immunoreactivity in C57B1/6 Mouse Brain Homogenates

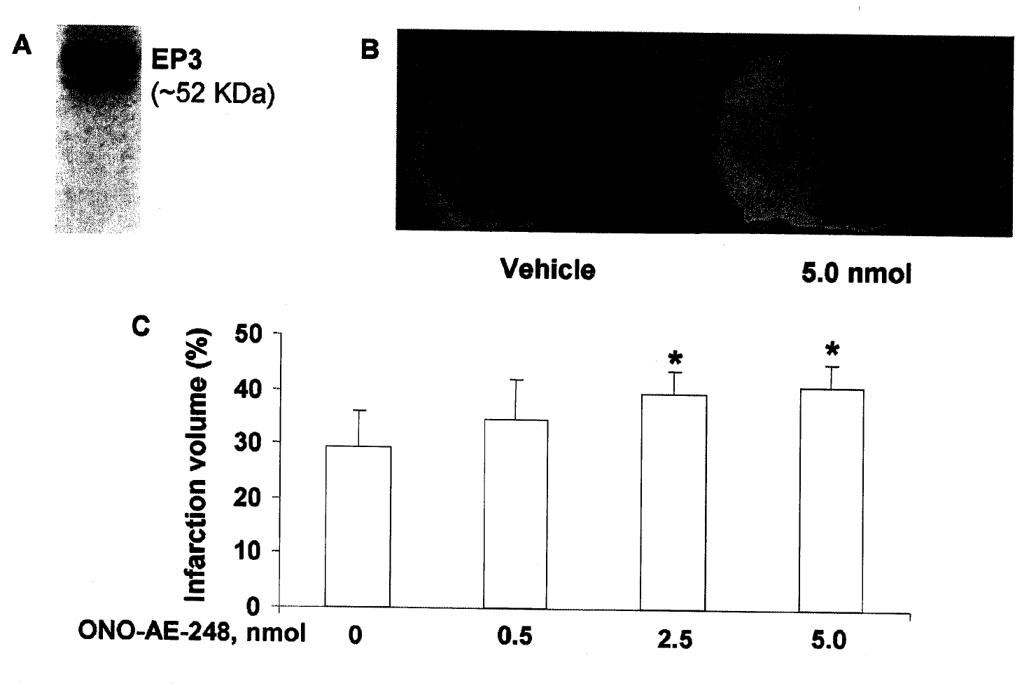

Western blot analysis of mouse brain homogenates revealed an immunoreactive band at the expected molecular weight for the mouse EP3 receptors (FIG. 2). The presence of EP3 receptors in the cerebrum is also supported by immunohistological and in situ hybridization studies in both rats and mice (Allen Brain Atlas, 2004; Engblom et al., 2004; Nakamura et al., 2000).

### EXAMPLE 3

### Effect of ONO-AE-248 on Infarct Volume

Intracerebroventricular (ICV) injection of ONO-AE-248 before MCAO significantly enhanced hemispheric infarct volumes. Infarct volume increased by 17%, 34%, and 38% in mice treated with 0.5-, 2.5-, and 5.0-nmol doses, respectively, as compared with the vehicle-treated group (n=9/dose). The increase was statistically significant only in the 2.5- and 5.0-nmol-treated groups (FIG. 2).

### EXAMPLE 4

### Cerebral Blood Flow

As estimated by laser-Doppler flowmetry, relative CBF decreased significantly in each group of mice after insertion of the nylon monofilament. Percent of baseline CBF was 18.0±3.3 for the vehicle-treated group and 18.0±3.7, 17.5±2.0, and 17.0±2.7 for the 0.5, 2.5, and 5.0 nmol-treated groups, respectively. The differences in CBF reduction were not significantly different.

### EXAMPLE 5

### Physiological Parameters

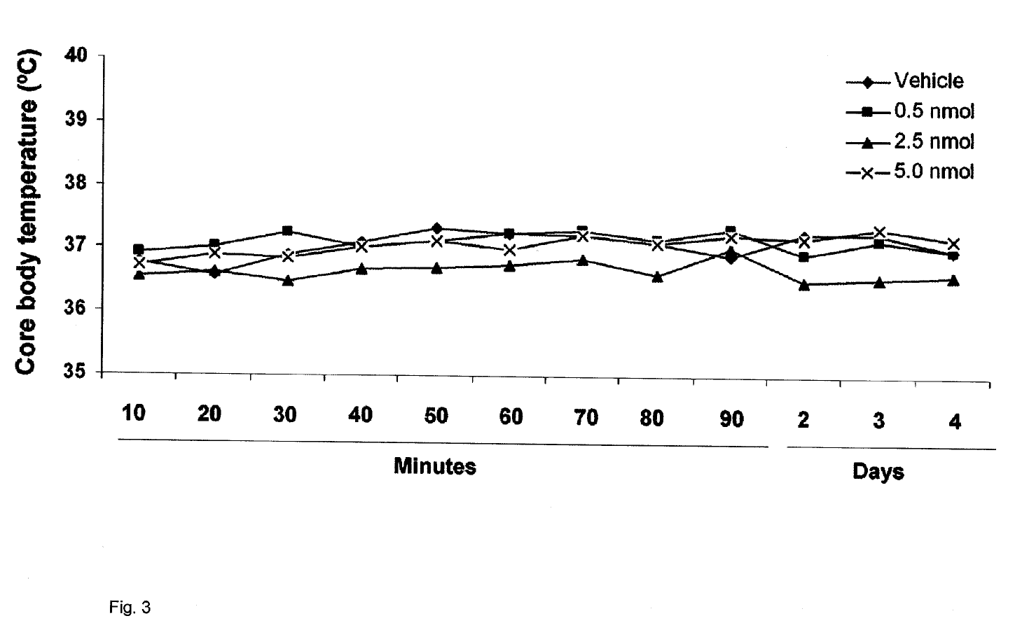

No statistically significant differences in pH, PaCO₂, PaO₂, or MABP were detected before ischemia, during ischemia, or during reperfusion among the groups of mice (Table 1). In a separate cohort of mice that did not undergo MCAO, we found that ONO-AE-248 did not affect core body temperature at any time during the 4-day experiment (FIG. 3).

### EXAMPLE 6

### EP3 Receptor Agonist ONO-AE-248 Exacerbates NMDA-Induced Brain Lesion

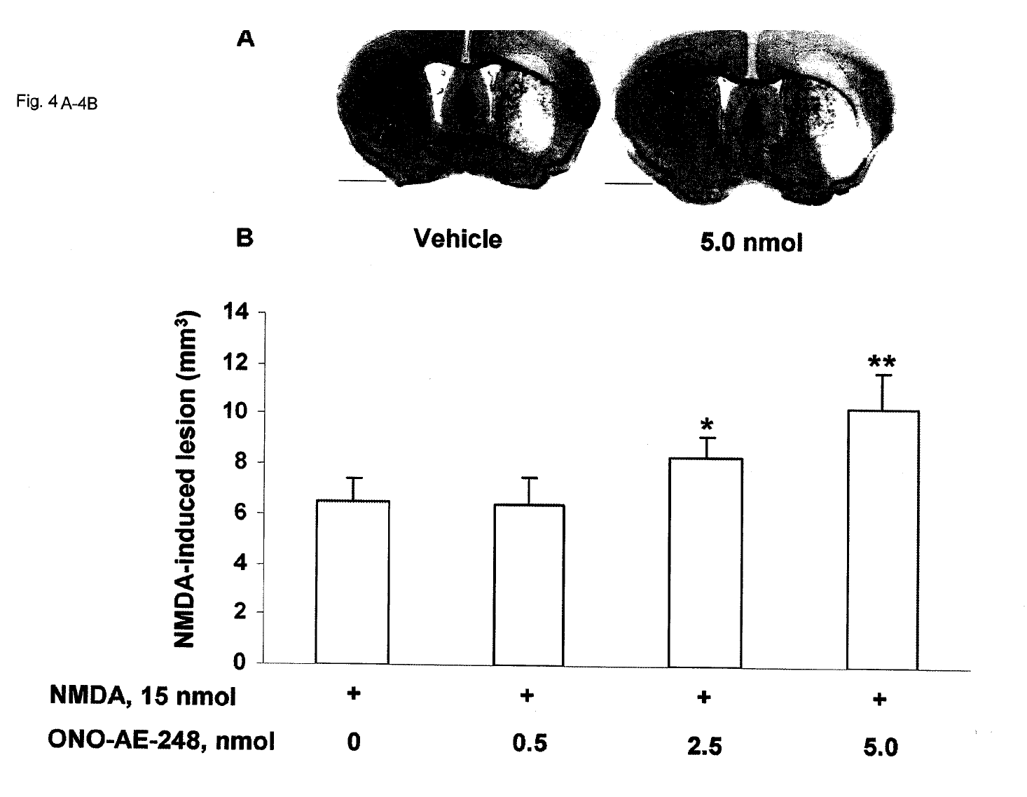

Pretreatment of mice with ONO-AE-248 aggravated the brain injury caused by NMDA injection. ONO-AE-248 significantly and dose-dependently increased the NMDA-induced lesion size in groups treated with 2.5 (p<0.02) and 5 nmol (p<0.001), whereas the lowest dose of ONO-AE-248 (0.5 nmol) did not affect NMDA-induced toxicity (FIG. 4).

### EXAMPLE 7

### Genetic Deletion of EP3 Receptor Protects Brain from Acute Excitotoxicity

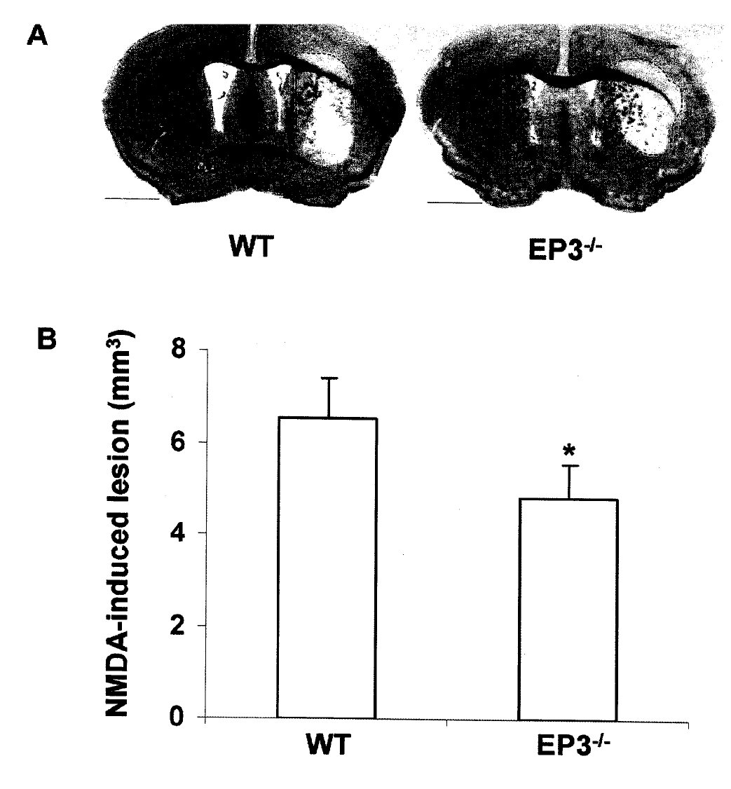

EP3^{-/-} mice were found to be less susceptible to NMDA-induced toxicity, than the WT mice (FIG. 5). The mean lesion volume in the NMDA-treated EP3^{-/-} mice was 26% smaller (p<0.01) than that of the NMDA-treated WT mice.

### EXAMPLE 8

### Genetic Deletion of EP3 Receptor Protects Brain from Ischemic Stroke

EP3^{-/-} mice that underwent transient ischemia had significantly (p<0.05) smaller infarct volumes than WT mice at 48 h after MCAO. Neurological score deficits correlated with infarct volume, but no significant differences in the physiological parameters monitored were detected between the two mouse strains.

For transient ischemia, the right middle cerebral artery (MCA) of wildtype (WT) and EP3 knockout (EP3^{-/-}) C57B1/6 mice was occluded with a nylon monofilament for 90 min and reperfused for 48 h, after which neurobehavioral scores and infarct volumes were determined. In another cohort of WT and EP3^{-/-} mice, mean arterial blood pressure, pH, blood gases (PaO₂ and PaCO₂), cerebral blood flow, and body temperature (measured by radio telemetry) were determined before and during reperfusion for 60 min. EP3 receptors in the brain have neuroprotective effect in focal cerebral ischemia.

### REFERENCES

The disclosure of each reference cited is expressly incorporated herein.
Ahmad, A. S., Ahmad, M., de Brum-Fernandes, A. J., Doré, S., 2005. Prostaglandin EP4 receptor agonist protects against acute neurotoxicity. Brain Res. 1066, 71-77.
Ahmad, A. S., Saleem, S., Ahmad, M., Doré, S., 2006a. Prostaglandin EP1 receptor contributes to excitotoxicity and focal ischemic brain damage. Toxicol. Sci. 89, 265-270.
Ahmad, A. S., Zhuang, H., Echeverria, V., Doré, S., 2006b. Stimulation of prostaglandin EP2 receptors prevents NMDA-induced excitotoxicity. J. Neurotrauma 23, 1895-1903.
Ahmad, M., Saleem, S., Zhuang, H., Ahmad, A. S., Echeverria, V., Sapirstein, A., Doré, S., 2006c. 1-HydroxyPGE₁ reduces infarction volume in mouse transient cerebral ischemia. Eur. J. Neurosci. 23, 35-42.
Aihara E, Nomura Y, Sasaki Y, Ise F, Kita K, Takeuchi K. Involvement of prostaglandin E receptor EP3 subtype in duodenal bicarbonate secretion in rats. Life Sci. 2007;80:2446-2453
Akerman, K. E., 1978. Changes in membrane potential during calcium ion influx and efflux across the mitochondrial membrane. Biochim. Biophys. Acta 502, 359-366.
Allen Brain Atlas [Internet]. 2004—cited Nov. 16, 2006. Available from: http://www.brain-map.org. Allen Institute for Brain Science, Seattle (Wash.).
Amano H, Hayashi I, Endo H, Kitasato H, Yamashina S, Maruyama T, Kobayashi M, Satoh K, Narita M, Sugimoto Y, Murata T, Yoshimura H, Narumiya S, Majima M. Host Prostaglandin E2-EP3 Signaling Regulates Tumor-Associated Angiogenesis and Tumor Growth 10.1084/jem.20021408. J Exp Med. 2003;197:221-232
Antonsson, B., Conti, F., Ciavatta, A., Montessuit, S., Lewis, S., Martinou, I., Bernasconi, L., Bernard, A., Mermod, J. J., Mazzei, G., Maundrell, K., Gambale, F., Sadoul, R., Martinou, J. C., 1997. Inhibition of Bax channel-forming activity by Bcl-2. Science 277, 370-372.
Atlante, A., Calissano, P., Bobba, A., Giannattasio, S., Marra, E., Passarella, S., 2001. Glutamate neurotoxicity, oxidative stress and mitochondria. FEBS Lett. 497, 1-5.
Bauer, M. K., Vogt, M., Los, M., Siegel, J., Wesselborg, S., Schulze-Osthoff, K., 1998. Role of reactive oxygen intermediates in activation-induced CD95 (APO-1/Fas) ligand expression. J. Biol. Chem. 273, 8048-8055.
Bilson, H. A., Mitchell, D. L., Ashby, B., 2004. Human prostaglandin EP3 receptor isoforms show different agonist-induced internalization patterns. FEBS Lett. 572, 271-275.
Collaco-Moraes, Y., Aspey, B., Harrison, M., de Belleroche, J., 1996. Cyclo-oxygenase-2 messenger RNA induction in focal cerebral ischemia. J. Cereb. Blood Flow Metab. 16, 1366-1372.
deCode genetics, I. DG041 for Peripheral arterial disease (PAD). 2006. http://www.decode.com/Pipeline/Index.php#pad. deCODE genetics, Inc., Reykjavik, Iceland.
Doré, S., Otsuka, T., Mito, T., Sugo, N., Hand, T., Wu, L., Hurn, P. D., Traystman, R. J., Andreasson, K., 2003. Neuronal overexpression of cyclooxygenase-2 increases cerebral infarction. Ann. Neurol. 54, 155-162.
Echeverria, V., Clerman, A., Doré, S., 2005. Stimulation of PGE₂ receptors EP2 and EP4 protects cultured neurons against oxidative stress and cell death following β-amyloid exposure. Eur. J. Neurosci. 22, 2199-2206.
Engblom, D., Ek, M., Ericsson-Dahlstrand, A., Blomqvist, A., 2004. EP3 and EP4 receptor mRNA expression in peptidergic cell groups of the rat parabrachial nucleus. Neuroscience 126, 989-999.
Fabre, J. E., Nguyen, M., Athirakul, K., Coggins, K., McNeish, J. D., Austin, S., Parise, L. K., FitzGerald, G. A., Coffman, T. M., Koller, B. H., 2001. Activation of the murine EP3 receptor for PGE₂ inhibits cAMP production and promotes platelet aggregation. J. Clin. Invest. 107, 603-610.
Goggel, R., Hoffman, S., Nusing, R., Narumiya, S., Uhlig, S., 2002. Platelet-activating factor-induced pulmonary edema is partly mediated by prostaglandin E(2), E-prostanoid 3-receptors, and potassium channels. Am J Respir Crit Care Med 166, 657-662.
Green, D. R., Reed, J. C., 1998. Mitochondria and apoptosis. Science 281, 1309-1312.
Hansen, M. R., Zha, X. M., Bok, J., Green, S. H., 2001. Multiple distinct signal pathways, including an autocrine neurotrophic mechanism, contribute to the survival-promoting effect of depolarization on spiral ganglion neurons in vitro. J. Neurosci. 21, 2256-2267.
Hayashi M, Kita K, Ohashi Y, Aihara E, Takeuchi K. Phosphodiesterase isozymes involved in regulation of HCO3-secretion in isolated mouse duodenum in vitro. Biochem Pharmacol. 2007;74:1507-1513
Huang, J., Upadhyay, U. M., Tamargo, R. J., 2006. Inflammation in stroke and focal cerebral ischemia. Surg. Neurol. 66, 232-245.
Iadecola, C., Niwa, K., Nogawa, S., Zhao, X., Nagayama, M., Araki, E., Morham, S., Ross, M. E., 2001. Reduced susceptibility to ischemic brain injury and N-methyl-D-aspartate-mediated neurotoxicity in cyclooxygenase-2-deficient mice. Proc. Natl. Acad. Sci. U.S.A. 98, 1294-1299.
Irie, A., Segi, E., Sugimoto, Y., Ichikawa, A., Negishi, M., 1994. Mouse prostaglandin E receptor EP3 subtype mediates calcium signals via Gi in cDNA-transfected Chinese hamster ovary cells. Biochem. Biophys. Res. Commun. 204, 303-309.
Jadhav, V., Jabre, A., Lin, S. Z., Lee, T. J., 2004. EP1- and EP3-receptors mediate prostaglandin E2-induced constriction of porcine large cerebral arteries. J. Cereb. Blood Flow Metab. 24, 1305-1316.
Kao, H. T., Song, H. J., Porton, B., Ming, G. L., Hoh, J., Abraham, M., Czernik, A. J., Pieribone, V. A., Poo, M. M., Greengard, P., 2002. A protein kinase A-dependent molecular switch in synapsins regulates neurite outgrowth. Nat. Neurosci. 5, 431-437.
Kawano, T., Anrather, J., Zhou, P., Park, L., Wang, G., Frys, K. A., Kunz, A., Cho, S., Orio, M., Iadecola, C., 2006. Prostaglandin E2 EP1 receptors: downstream effectors of COX-2 neurotoxicity. Nat. Med. 12, 225-229.
Kiriyama, M., Ushikubi, F., Kobayashi, T., Hirata, M., Sugimoto, Y., Narumiya, S., 1997. Ligand binding specificities of the eight types and subtypes of the mouse prostanoid receptors expressed in Chinese hamster ovary cells. Br. J. Pharmacol. 122, 217-224.
Lipton, P., 1999. Ischemic cell death in brain neurons. Physiol. Rev. 79, 1431-1568.
Loew, L. M., Carrington, W., Tuft, R. A., Fay, F. S., 1994. Physiological cytosolic Ca2+ transients evoke concurrent mitochondrial depolarizations. Proc. Natl. Acad. Sci. U.S.A. 91, 12579-12583.
Lucas, S. M., Rothwell, N. J., Gibson, R. M., 2006. The role of inflammation in CNS injury and disease. Br. J. Pharmacol. 147 Suppl 1, S232-240.
Martin, M., Meyer-Kirchrath, J., Kaber, G., Jacoby, C., Flogel, U., Schrader, J., Ruther, U., Schror, K., Hohlfeld, T., 2005. Cardiospecific overexpression of the prostaglandin EP3 receptor attenuates ischemia-induced myocardial injury. Circulation 112, 400-406.
Minghetti, L., 2004. Cyclooxygenase-2 (COX-2) in inflammatory and degenerative brain diseases. J. Neuropathol. Exp. Neurol. 63, 901-910.
Morham, S. G., Langenbach, R., Loftin, C. D., Tiano, H. F., Vouloumanos, N., Jennette, J. C., Mahler, J. F., Kluckman, K. D., Ledford, A., Lee, C. A., Smithies, O., 1995. Prostaglandin synthase 2 gene disruption causes severe renal pathology in the mouse. Cell 83, 473-482.
Nakamura, K., Kaneko, T., Yamashita, Y., Hasegawa, H., Katoh, H., Negishi, M., 2000. Immunohistochemical localization of prostaglandin EP3 receptor in the rat nervous system. J Comp Neurol 421, 543-569.
Namba, T., Sugimoto, Y., Negishi, M., Irie, A., Ushikubi, F., Kakizuka, A., Ito, S., Ichikawa, A., Narumiya, S., 1993. Alternative splicing of C-terminal tail of prostaglandin E receptor subtype EP3 determines G-protein specificity. Nature 365, 166-170.
Narumiya, S., FitzGerald, G. A., 2001. Genetic and pharmacological analysis of prostanoid receptor function. J. Clin. Invest. 108, 25-30.
Narumiya, S., Sugimoto, Y., Ushikubi, F., 1999. Prostanoid receptors: structures, properties, and functions. Physiol. Rev. 79, 1193-1226.
Norel, X., de Montpreville, V., Brink, C., 2004. Vasoconstriction induced by activation of EP1 and EP3 receptors in human lung: effects of ONO-AE-248, ONO-DI-004, ONO-8711 or ONO-8713. Prostaglandins Other Lipid Mediat. 74, 101-112.
Oka, T., 2004. Prostaglandin E2 as a mediator of fever: the role of prostaglandin E (EP) receptors. Front. Biosci. 9, 3046-3057.
Planas, A. M., Soriano, M. A., Rodriguez-Farre, E., Ferrer, I., 1995. Induction of cyclooxygenase-2 mRNA and protein following transient focal ischemia in the rat brain. Neurosci. Lett. 200, 187-190.
Rydel, R. E., Greene, L. A., 1988. cAMP analogs promote survival and neurite outgrowth in cultures of rat sympathetic and sensory neurons independently of nerve growth factor. Proc. Natl. Acad. Sci. U.S.A. 85, 1257-1261.
Schinder, A. F., Olson, E. C., Spitzer, N. C., Montal, M., 1996. Mitochondrial dysfunction is a primary event in glutamate neurotoxicity. J. Neurosci. 16, 6125-6133.
Shah, Z. A., Namiranian, K., Klaus, J., Kibler, K., Dore, S., 2006. Use of an optimized transient occlusion of the middle cerebral artery protocol for the mouse stroke model. J Stroke Cerebrovasc Dis 15, 133-138.
Slawik, H., Volk, B., Fiebich, B., Hull, M., 2004. Microglial expression of prostaglandin EP3 receptor in excitotoxic lesions in the rat striatum. Neurochem. Int. 45, 653-660.
Sugimoto, Y., Negishi, M., Hayashi, Y., Namba, T., Honda, A., Watabe, A., Hirata, M., Narumiya, S., Ichikawa, A., 1993. Two isoforms of the EP3 receptor with different carboxyl-terminal domains. Identical ligand binding properties and different coupling properties with Gi proteins. J. Biol. Chem. 268, 2712-2718.
Suzawa, T., Miyaura, C., Inada, M., Maruyama, T., Sugimoto, Y., Ushikubi, F., Ichikawa, A., Narumiya, S., Suda, T., 2000. The role of prostaglandin E receptor subtypes (EP1, EP2, EP3, and EP4) in bone resorption: an analysis using specific agonists for the respective EPs. Endocrinology 141, 1554-1559.
Tomimoto, H., Shibata, M., Ihara, M., Akiguchi, I., Ohtani, R., Budka, H., 2002. A comparative study on the expression of cyclooxygenase and 5-lipoxygenase during cerebral ischemia in humans. Acta Neuropathol. (Berl) 104, 601-607.
Waldner, C., Heise, G., Schror, K., Heering, P., 2003. COX-2 inhibition and prostaglandin receptors in experimental nephritis. Eur J Clin Invest 33, 969-975.
Yamazaki, K., Endo, T., Kitajima, Y., Manase, K., Nagasawa, K., Honnma, H., Hayashi, T., Kudo, R., Saito, T., 2005. Elevation of both cyclooxygenase-2 and prostaglandin E(2) receptor EP3 expressions in rat placenta after uterine artery ischemia-reperfusion. Placenta.
Yuhki, K., Ueno, A., Naraba, H., Kojima, F., Ushikubi, F., Narumiya, S., Oh-ishi, S., 2004. Prostaglandin receptors EP2, EP3, and IP mediate exudate formation in carrageenin-induced mouse pleurisy. J. Pharmacol. Exp. Ther. 311, 1218-1224.
Zacharowski, K., Olbrich, A., Piper, J., Hafner, G., Kondo, K., Thiemermann, C., 1999. Selective activation of the prostanoid EP(3) receptor reduces myocardial infarct size in rodents. Arterioscler Thromb Vasc Biol 19, 2141-2147.
Zamzami, N., Marchetti, P., Castedo, M., Decaudin, D., Macho, A., Hirsch, T., Susin, S. A., Petit, P. X., Mignotte, B., Kroemer, G., 1995. Sequential reduction of mitochondrial transmembrane potential and generation of reactive oxygen species in early programmed cell death. J. Exp. Med. 182, 367-377.

We claim:

1. A method of treating a patient with an acute or chronic neurodegenerative disease, comprising:
administering to the patient an antagonist of an EP3 receptor, whereby a symptom of the disease is ameliorated.

2. The method of claim 1 wherein the patient has Alzheimer's disease.

3. The method of claim 1 wherein the patient has Parkinson's disease.

4. The method of claim 1 wherein the patient has Huntington's disease.

5. The method of claim 1 wherein the patient has had an ischemic stroke.

6. The method of claim 1 wherein the patient has had a hemorrhagic stroke.

7. The method of claim 1 wherein the patient has global ischemia.

8. The method of claim 1 wherein the patient has head trauma.

9. The method of claim 1 wherein the patient has age-related vascular dementia.

10. The method of claim 1 wherein the patient has a cognitive disorder.

11. The method of claim 1 wherein the patient does not have peripheral arterial disease.

12. The method of claim 1 wherein the antagonist is delivered intracerebroventricularly.

13. The method of claim 1 wherein the antagonist is delivered per os.

14. The method of claim 1 wherein the antagonist is delivered intraperitoneally.

15. The method of claim 1 wherein the antagonist is delivered intravenously.

16. The method of claim 1 wherein the antagonist is selected from the group consisting of an antibody, antibody fragment, single-chain antibody, chimeric antibody, humanized antibody, and human antibody.

17. The method of claim 1 wherein the antagonist is selected from the group consisting of EP3-P, EP3-N, L-798106, ONO-AE3-240, ONO-AE3-208, ONO 8711, SC-51322, and DG041.

18. The method of claim 1 wherein the patient does not have peripheral arterial disease.

19. A method of reducing the risk of an ischemic reperfusion event in a subject, comprising:
administering to a subject at increased risk of having a stroke an antagonist of an EP3 receptor, whereby the risk of a stroke is reduced.

20. The method of claim 19 wherein the subject has already had a stroke.

21. The method of claim 19 wherein the patient is genetically prone to stroke.

22. The method of claim 19 wherein the antagonist is delivered intracerebroventricularly.

23. The method of claim 19 wherein the antagonist is delivered per os.

24. The method of claim 19 wherein the antagonist is delivered intraperitoneally.

25. The method of claim 19 wherein the antagonist is delivered intravenously.

26. The method of claim 19 wherein the antagonist is selected from the group consisting of an antibody, antibody fragment, single-chain antibody, chimeric antibody, humanized antibody, and human antibody.

27. The method of claim 19 wherein the antagonist is selected from the group consisting of EP3-P, EP3-N, L-798106, ONO-AE3-240, ONO-AE3-208, ONO 8711, SC-51322, and DG041.

28. The method of claim 19 wherein the patient does not have peripheral arterial disease.

29. The method of claim 19 wherein the patient is prone to stroke due to a pharmacological intervention

30. The method of claim 19 wherein the patient is prone to stroke due to a surgical intervention.
